(19) United States
(12) Patent Application Publication
(10) Pub. No.: US 2009/0165360 A1
(43) Pub. Date: Jul. 2, 2009

(54) FRAGRANCE FIXATIVES

(75) Inventors: Suzanne Winegar Dobbs, Kingsport, TN (US); Terry Ann Oldfield, Kingsport, TN (US); David Justin Olsen, Kingsport, TN (US)

Correspondence Address: William K. McGreevey;Eastman Chemical Company, P.O. Box 511, Kingsport, TN, 37662, US

(73) Assignee: EASTMAN CHEMICAL COMPANY, Kingsport, TN (US)

(21) Appl. No.: 12/330,532

(22) Filed: Dec. 9, 2008

Related U.S. Application Data
(60) Provisional application No. 61/016,885, filed on Dec. 27, 2007.

(51) Int. Cl. C11C 5/00; A61L 9/02; C10L 5/00

(52) U.S. Cl. 44/275; 512/4

(57) ABSTRACT

Compositions are provided that contain fragrance compounds and fragrance fixatives selected from diesters of TMPD. Scented compositions and scented articles containing such compositions are also provided. Also disclosed are methods for imparting a fragrance to a location and methods for formulating the compositions of the present invention.

### CROSS REFERENCE TO RELATED APPLICATIONS

This application claims the benefit of U.S. Provisional Application Ser. No. 61/016,885 filed on Dec. 27, 2007, the disclosure of which is incorporated herein by reference to the extent it does not contradict the disclosures herein.

### FIELD OF THE INVENTION

The present invention relates to fragrance compositions. More particularly, the present invention relates to fragrance compositions that contain at least one fragrance compound and at least one diester of 2,2,4-trimethyl-1,3-pentane diol (TMPD).

### BACKGROUND OF THE INVENTION

The importance of fragrances and perfumes has been understood for hundreds of years. Fragrances and perfumes have been and continue to be utilized for personal and commercial uses. To slow the evaporation of fragrance compounds, the fragrance compounds are sometimes combined with lower volatility components generally known as fixatives. These fixatives are substances which improve lasting qualities of odorous substances of a fragrance. There is a continuing need for the development of new fragrance fixatives.

### SUMMARY OF THE INVENTION

The present invention is based on the discovery that the use of diesters of 2,2,4-trimethyl-1,3-pentane diol (TMPD) in perfumes or fragrances can provide a more enduring fragrance by slowing the evaporation of fragrance compounds. The invention therefore provides compositions that contain at least one fragrance compound and at least one diester of TMPD. The invention further provides articles that contain the compositions of the present invention. The invention further provides methods of formulating and using the compositions of a present invention.

Thus, the invention provides fragrance compositions that result in longer lasting aromas. In some embodiments, the fragrance composition contains at least one fragrance compound and at least one diester of TMPD. In some embodiments, the diester of TMPD is selected from TMPD di-2-ethyl hexanoate, 2,2,4-trimethyl-1,3-pentanediol di-isobutyrate (available as TXIB from Eastman Chemical Company) TEXANOL laurate, and TMPD di-laurate. In some embodiments, the diester of TMPD is TXIB or TMPD di-2-ethyl hexanoate. In some embodiments, the diester of TMPD is TMPD di-2-ethyl hexanoate.

The invention further comprises scented compositions. In some embodiments, the scented compositions contain a fragrance composition of the present invention and at least one additional component. In some embodiments, the scented compositions contain at least one fragrance compound, at least one wax, and at least one diester of TMPD. Some scented compositions are solid at room temperature (25° C.) and standard pressure (1 atmosphere). For example, the invention provides a solid scented composition containing between about 0.5% and about 10% by weight of at least one fragrance compound, one or more waxes present in an amount sufficient to render the solid scented composition a solid at room temperature and standard pressure, and between about 0.5% and about 15% by weight of at least one diester of TMPD. Some scented compositions are liquid at standard pressure and room temperature.

The invention also provides articles that include or contain the fragrance compositions or the scented compositions of the present invention. In some embodiments, the article is a solid scented object such as a candle or a solid air freshener.

The invention also provides methods of imparting an aroma to a location, in which a fragrance composition or scented composition of the present invention is delivered to the location.

The invention also provides methods for formulating fragrance compositions, in which at least one fragrance compound and at least one diester of TMPD are combined. In some embodiments, the diester of TMPD is selected from TMPD di-2-ethyl hexanoate, 2,2,4-trimethyl-1,3-pentanediol di-isobutyrate (available as TXIB from Eastman Chemical Company) TEXANOL laurate (which can by made, for example, though an esterification reaction between 2,2,4-trimethyl-1,3-pentanediol monoisobutyrate and lauric acid), and TMPD di-laurate. In some embodiments, the diester of TMPD is TXIB or TMPD di-2-ethyl hexanoate. In some embodiments, the diester of TMPD is TMPD di-2-ethyl hexanoate.

The invention also provides methods for formulating scented compositions, in which at a fragrance composition and an additional component are combined.

### DETAILED DESCRIPTION OF THE INVENTION

The invention provides compositions that contain at least one fragrance compound and at least one diester of 2,2,4-trimethyl-1,3-pentane diol (TMPD). The invention further provides scented compositions and scented articles that contain the compositions of the present invention. The invention further provides methods of delivering fragrance compositions to a selected location as well as methods of formulating the fragrance compositions.

Compositions

The compositions of the present invention contain at least one fragrance compound and at least one diester of TMPD. In some embodiments, the composition is a fragrance composition containing at least one fragrance compound and at least one diester of TMPD. In some embodiments, the composition is a scented composition that contains a fragrance composition of the present invention. Such compositions can optionally contain any other desired components.

Fragrance Compositions and Compounds

As used throughout this application, “fragrance compound” means any compound that possesses an aroma that is detectable to human olfactory senses when in a free or un-entrapped state at room temperature (about 25 degrees C.). Some examples of “fragrance compounds” include natural oils and other natural materials, synthetic oils, alcohols, aldehydes, ketones, esters, terpene compounds, carboxylic acids, lactones, nitrogenous or sulfurous heterocyclic compounds, ethers, hydrocarbons, nitrites and other classes of chemical compounds. Many of these ingredients are listed in reference texts such as “Perfume and Flavor Chemicals (Aroma Chemicals),” Steffen Arctander, published by the author, 1969 Montclair, N.J., “Perfumery, Practice and Principles” Robert R. Calkin and J. Stephan Jellinek, John Wiley & Sons, Inc. 1994, or in other works of a similar nature.

As used throughout this application, “fragrance composition” shall mean any composition that contains one or more fragrance compounds. Fragrance compositions release fragrance compounds upon delivery to a desired location under desired conditions, in amounts sufficient to produce a desired aroma. In some embodiments, fragrance compositions contain several fragrance compounds or several combinations of fragrance compounds having different release profiles after application.

In some embodiments, the fragrance compositions contain different “notes,” typically referred to as: “top note,” denoting the aroma that is most noticeable immediately after delivery or application of a fragrance composition and caused or influenced by the fragrance compounds that are most volatile or otherwise release the most quickly from the composition; “middle note,” an intermediate aroma that bridges from top note to the base or bottom note and due to fragrance compounds having an intermediate release profile; and the “base note” or “bottom note,” which are those materials which have an aroma that is detectable the longest after application. In some embodiments, a formulation containing top, middle and base notes is prepared to give a desired balance between these three groups. Mixtures of fragrance materials are known by those skilled in the art of fragrances and perfumes as “accords.”

A fragrance composition may contain any desired combination of fragrance compounds and resulting notes, formulated to achieve desired fragrance character. The fragrance composition can, for example, include ingredients providing various notes of the fragrance families (for example, green notes, fruity notes, aldehydic notes, chypre notes, oriental notes, tobacco notes, leather notes and fougere notes), and sub-classifications thereof (such as fresh and balsamic green notes; fresh and sweet floral notes; floral and floral-woody-powdery aldehydic notes; and fresh-mossy-aldehydic, floral-mossy-animalic and mossy-fruity chypre notes). Any acceptable fragrance compounds or combinations thereof can be utilized. Moreover, the existence of a certain number of notes not critical to the invention and fragrances having any number of one or more notes are within the scope of the invention.

Diesters of TMPD

As used throughout this application, “diester of TMPD” or “diester of 2,2,4-trimethyl-1,3-pentane diol” means a compound having a structure described by Formula I:

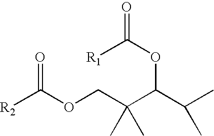

wherein R₁ and R₂ each independently represent a C₃ to C₁₇ alkyl group. By “independently,” it is mean that R₁ and R₂ may be the same or different. Although any such compounds are within the present invention irrespective of how made or derived, one method of making such compound is through esterification of the alcohol groups of 2,2,4-trimethyl-1,3-pentane diol (TMPD) or of a monoester of TMPD such as 2,2,4-trimethyl-1,3-pentanediol monoisobutyrate (available as TEXANOL from Eastman Chemical Company). In some embodiments, the esterification is through the reaction of the alcohol groups with carboxylic acid groups. For example, an esterification reaction of one mole of TMPD with two moles of 2-ethyl hexanoic acid would yield TMPD di-2-hexanoate, in which R₁ and R₂ are each C₇ linear chains in which the third carbon is bonded to the carbon in the ester linkage.

In some embodiments, the diester of TMPD is selected from TMPD di-2-ethyl hexanoate, 2,2,4-trimethyl-1,3-pentanediol di-isobutyrate (available as TXIB from Eastman Chemical Company) TEXANOL laurate, and TMPD di-laurate. Embodiments involving any one of the foregoing diesters of TMPD or combinations of two or more of the foregoing diesters of TMPD are also within the present invention. Thus, in some embodiments, the diester of TMPD is TXIB; in some embodiments, the diester of TMPD is TMPD di-2-ethyl hexanoate.

The diester of TMPD may be present in any acceptable amount in the fragrance compositions of the present invention. In some embodiments, the diester of TMPD is present in a fragrance composition in a range of from about 0.01 to about 95 weight %, based on the total weight of the composition. In some embodiments, the diester of TMPD is present in an amount in a narrower range, such as from about 0.01 to about 90 weight %, from about 0.05 to about 40 weight %, from about 0.5 to about 25 weight %, from about 0.5 to about 15 weight %, from about 0.5 to about 10 weight %, from about 25 to about 50 weight %, from about 50 to about 75 weight %, from about 75 to about 99.5 weight %, from about 55 to about 95 weight %, from about 50 to about 99.5 weight %, from about 5 to about 10 weight %, from about 40 to about 80 weight %, from about 60 to about 80 weight %, and from about 85 to about 95 weight %. In some embodiments, the amount of diester of TMPD ranges from about 50 to about 75 weight %. Depending upon the specific formulation for the fragrance composition and a resulting scented composition, narrower embodiments also exist in which the range is selected from 0.1-10, 10-20, 20-30, 30-40, 40-50, 50-60, 60-70, 70-80, 80-90, 90-99.5% by weight, or a large range based on combining two or more of such ranges. In some embodiments, the amount of diester of TMPD is between about 0.1 to 15% by weight. In some embodiments the foregoing percentages are based on the weight of the fragrance composition. In some embodiments the foregoing percentages are based on the weight of an entire scented composition as defined below.

In some embodiments, at least one diester of TMPD in the composition has a Total Hansen Solubility Parameter (also referred to as the Hildebrand Solubility Parameter) within 5.0 units of the Total Hansen Solubility Parameter of one or more fragrance compound(s) in the composition. In some embodiments, at least one diester of TMPD in the composition has a Total Hansen Solubility Parameter within 4.0 units of the Total Hansen Solubility Parameter of one or more fragrance compound(s) in the composition. In some embodiments, at least one diester of TMPD in the composition has a Total Hansen Solubility Parameter within 3.0 units of the Total Hansen Solubility Parameter of one or more fragrance compound(s) in the composition. In some embodiments, at least one diester of TMPD in the composition has a Total Hansen Solubility Parameter within 2.0 units of the Total Hansen Solubility Parameter of one or more fragrance compound(s). In some embodiments, at least one diester of TMPD in the composition has a Total Hansen Solubility Parameter within 1.0 units of the Total Hansen Solubility Parameter of one or more fragrance compound(s). As used herein, the “Total Hansen Solubility Parameter” refers to the amount estimated (in units of (calories/cubic centimeter)^{1/2}) using the group-contribution method described in “Hansen Solubility Parameters—A User's Handbook” by C. M. Hansen, pp. 9-10, 167-185, CRC Press, Boca Raton, Fla., 2000. The Total Hansen Solubility Parameter (or Hildebrand Solubility Parameter) is made up of three components: a dispersion force component, a polar component, and a hydrogen bonding component. It is calculated from the heat of vaporization using the formula:

Total Solubility Parameter=((HV−RT)/LVOL)^{1/2}

where HV=Molar Heat of Vaporization, R=Gas Constant, T=Absolute Temperature, and LVOL=Liquid Molar Volume at T.

Using the above methods, the Hildebrand Solubility Parameter for TXIB, TMPD di-2-ethyl hexanoate, and TEXANOL laurate were each calculated to be 8.3 (cal/cm³)^{1/2} and the total solubility parameter for TMPD di-laurate was calculated to be 8.2 (cal/cm³)^{1/2}.

By way of comparison, Hildebrand solubility parameters for the following examples of fragrance compounds, are published in Chemistry and Technology of Flavors and Fragrance, edited by David Rowe, “Chapter 13, Applications II: Fragrance” by Stephen J. Herman, Blackwell Publishing (2004), p 310 as follows: isoamyl acetate, 8.4 (cal/cc)^{1/2}; citronellal, 8.8 (cal/cc)^{1/2}; benzyl alcohol, 12.3 (cal/cc)^{1/2}; linalool, 9.6 (cal/cc)^{1/2}; and citronellol, 9.9 (cal/cc)^{1/2}. In some embodiments, all of the individual Hansen Solubility Parameters for both the diester of TMPD and the fragrance compound in the composition are within one of the ranges specified in this paragraph. In some embodiments, one, two, or three of the individual Hansen Solubility Parameters are within one of the ranges specified in this paragraph.

Other Components in the Fragrance Composition

The perfume or fragrance and the fixatives according to the present invention may be used alone or with other perfuming, non-perfuming or active ingredients. The compositions of the present invention can contain other components such as solvents, preservatives, antioxidants, additional fixatives, extenders, stabilizers, UV screening agents and the like. Some examples of useful solvents include ethanol, water/ethanol mixtures; isopropanol; diethylene glycol monoethyl ether; glycerol, propylene glycol, 1,2-butyleneglycol, dipropylene glycol, diethyl phthalate, ethyl citrate (2-(2-ethoxyethoxy)-1-ethanol), triethyl citrate, isopropyl myristate, waxes, isoparaffins, glycol ethers and glycol ether esters.

Scented Compositions

The invention also includes scented compositions that contain at least one fragrance compound, at least one diester of TMPD, and at least one additional component. In some embodiments, the scented compositions contain a fragrance composition of the present invention along with at least one component, obtainable, for example by combining at least one fragrance composition and at least one additional component. In some embodiments, the scented compositions are obtained by combining all three components simultaneously. Some scented compositions are solid at room temperature and standard pressure. Some are liquid at room temperature and standard pressure. In some embodiments, the scented compositions contain at least one wax.

Scented compositions include many fully formulated commercial products or fully formulated parts of commercial products. Some examples of such products include eau de parfum, eau de toilette, aftershave and preshave products, eau de colognes, splash colognes, perfumed freshening wipes, perfuming neutral cleaners (e.g. floor cleaners, window cleaners, dishwashing detergents, bath and sanitary cleaners, carpet cleaner foams and powders, liquid detergents, detergent powders, laundry pretreatment agents such as bleaches, soaking agents and stain removers), fabric softeners, washing soaps, washing tablets, disinfectants, air fresheners or scented portions thereof (in liquid, gel or solid form), aerosol sprays, waxes and polishes, (e.g. furniture polishes, floor waxes, shoe creams), solid and liquid soaps, shower gels, shampoos, shaving soaps and creams, bath oils, cosmetic emulsions (e.g. skin creams and lotions, face creams and lotions, sunscreen creams and lotions, after sun creams and lotions, hand creams and lotions, foot creams and lotions, depilatory creams and lotions) hair sprays, hair gels, hair setting lotions, hair rinses, hair dyes and colorants, hair-shaping compositions, hair-smoothing compositions, hair tonics, hair creams and lotions, deodorants and antiperspirants (e.g. sprays, sticks and roll-ons), deodorant creams, decorative cosmetics (e.g., eye shadow, nail varnish, make-up, lipsticks, mascara), candles and candle wax materials, lamp oils, joss-sticks, furniture sprays, insecticides, and insect repellents.

Additional components added to scented compositions can include colorants, waxes, antibacterial agents, antifungal agents, gelling agents (e.g. metallic soaps such as sodium stearate and/or sodium isostearate), dibenzylidene sorbitol or a mixed glycol system in combination with dibenzylidene sorbitol), anti-irritants, emollients, surfactants, abrasives, absorbents, anti-caking agents, anti-oxidants, vitamins, binders, biological additives, buffering agents, bulking agents, chelating agents, chemical additives, cosmetic astringents, cosmetic biocides, denaturants, drug astringents, external analgesics, film formers, humectants, opacifying agents, pH adjusters, preservatives, propellants, reducing agents and skin bleaching agents. In some embodiments, the fragrance composition can be adsorbed on a carrier which serves to distribute the fragrance finely, to release it in a controlled manner during use, or both. Some examples of such carriers include sulfate, silica gels, zeolites, gypsums, clays, clay granules, gas concrete or organic materials such as woods, cellulose-based substances and their derivatives, polymers and plastics. The foregoing list is not limited and scented compositions can comprise any optional components. Several such components are well known in the art. The CTFA International Cosmetic Ingredient Dictionary and Handbook, Eleventh Edition, 2006, for example, describes many examples of ingredients commonly used in the skin and hair care industry.

Some embodiments of scented compositions, for example some solid compositions, contain one or more waxes. As used in this application, a “wax” refers to any of the high molecular weight organic compounds or mixtures that are solid at room temperature and standard pressure and that have a reversible melting point range of from 40° to 120° C. (“reversible” meaning that the solid becomes a liquid upon heating and returns to a solid upon cooling). Examples include certain hydrocarbons and fatty acid esters and combinations thereof. Some specific examples of waxes include waxes of mineral or petroleum derivations (e.g. montan or lignite wax, paraffin wax, cerisin, ozokerite, and microcrystalline wax); waxes of plant derivation (e.g. bayberry wax, carnauba wax, candelilla wax, Japan wax, jojoba wax, bayberry wax, castor wax, soy wax, palm wax, and rice bran wax) waxes of animal derivation (e.g. include beeswax, lanolin chinese insect wax, shellac wax, and spermaceti wax) and synthetic waxes (e.g. polyethylene wax and polymerized alpha olefin wax, chlorinated napthalenes and certain polyol ether-esters). In some embodiments, the wax is a paraffin wax or beeswax. In some embodiments, the wax is a paraffin wax. In some embodiments, the wax is present in an amount of at least about 50% by weight based on the total weight of the scented composition. In some embodiments, the wax is present in an amount of at least about 70% by weight based on the total weight of the scented composition. In some embodiments, the wax is present in an amount of at least about 80% by weight based on the total weight of the scented composition.

In some embodiments involving waxes, additives used include compounds that harden the composition, increase its melting point, or both. Some examples include long-chain fatty acid or additional waxes. Such additives can be useful in embodiments involving waxes that are softer or have a lower melting point than is desired in the final compositions. In some embodiments, such additives are present in concentrations up to about 20%. In some embodiments, such additives are present in concentrations up to about 30%. In some embodiments, at least one additive is a fatty acid having 16-20 carbons. In some embodiments, at least one additive is stearic acid.

As noted above, one way of preparing scented compositions is to first prepare a fragrance composition of the present invention, then combine the fragrance composition with one or more additional components. However, the scented compositions may be made by combining the components in any order that is effective to combine them (e.g., simultaneously, combine the diester of TMPD and additional components first, etc.). Thus, although proportions of fragrance compositions in the scented compositions are discussed below, the invention is not limited to compositions that are prepared by formulating the fragrance compositions first. The proportions in fragrance compositions of the present invention can be incorporated into scented compositions at a broad range of concentrations. These values depend on the nature of the product to be perfumed and on the olfactory effect sought, as well as on the nature of the co-ingredients in a given composition when the compounds of the invention are used in admixture with perfuming co-ingredients, solvents or additives commonly used in the art. In some embodiments, the amount of fragrance composition is between about 1 and about 50 weight % of the scented composition based on the total weight of the scented composition. In some embodiments, the weight range for fragrance compositions is a narrower range, such as about 0.1 to about 40% by weight, about 0.5 to about 20% by weight, about 5 to about 10% by weight, about 0.5 to about 10% by weight, about 1 to about 15% by weight, about 5 to about 15% by weight, about 15 to about 30% by weight, about 1 to about 5% by weight, about 0.1 to about 2% by weight, and so on, in each case the percentages being based on the weight of the total product.

Percentages of the diester of TMPD in the scented composition from various embodiments are discussed above in the description of fragrance compositions.

Articles

The invention further comprises scented articles that comprise one or more compositions of the present invention. The articles comprise one or more fragrance compositions, scented compositions, or both. Some examples include air fresheners, candles, packaging, solid compositions of the present inventions, and articles made from scented polymers (e.g. writing implements, toys, films).

Methods

The invention further includes methods for imparting an aroma to a location. The method involves delivering a fragrance composition or scented composition of the present invention to the location. The compositions may be delivered by any means including, but not limited to spraying, brushing on, pouring on, dipping or immersing, applying by hand or with a contact applicator, dripping application, or simply placing a solid scented composition in a desired location.

The invention further includes methods for formulating the fragrance compositions of the present invention. The methods include combining at least one fragrance compound with at least one diester of TMPD. In some embodiments, the diester of TMPD is selected from TMPD di-2-ethyl hexanoate, 2,2,4-trimethyl-1,3-pentanediol di-isobutyrate (available as TXIB from Eastman Chemical Company) TEXANOL laurate, and TMPD di-laurate. In some embodiments, the diester of TMPD is TXIB or TMPD di-2-ethyl hexanoate. In some embodiments, the diester of TMPD is TMPD di-2-ethyl hexanoate.

The invention further includes methods for formulating the scented compositions of the present invention. The methods include combining at least one fragrance compound, at least one diester of TMPD, and at least one additional component, in any desired order. In some embodiments, the diester of TMPD and fragrance compound are first combined together, then combined with the additional components. In some embodiments, the diester of TMPD is selected from TMPD di-2-ethyl hexanoate, 2,2,4-trimethyl-1,3-pentanediol di-isobutyrate (available as TXIB from Eastman Chemical Company) TEXANOL laurate, and TMPD di-laurate. In some embodiments, the diester of TMPD is TXIB or TMPD di-2-ethyl hexanoate. In some embodiments, the diester of TMPD is TMPD di-2-ethyl hexanoate.

The present invention is illustrated in greater detail by the specific examples presented below. It is to be understood that these examples are illustrative embodiments and are not intended to be limiting of the invention, but rather are to be construed broadly within the scope and content of the appended claims. All parts and percentages in the examples are on a weight basis unless otherwise stated.

### EXAMPLE 1

### Effect of Fixatives on Evaporation Rate of Fragrance Components as Determined by Weight Loss Over Time

Separate mixtures of various individual fixatives (TXIB, TMPD di-2-ethyl hexanoate, TEXANOL laurate and TMPD di-laurate, as well as diethyl phthalate as a control) with various individual aroma chemicals were prepared by weighing the liquids into vials. Into each vial was weighed 90 g of fixative and 10 g of an aroma chemical (isoamyl acetate, benzyl alcohol, linalool, or citronellol), so that the weight ratio of fixative to aroma chemical was 9:1. Vials were then sealed. Each mixture of fixative and aroma chemical was stirred and observed to confirm that the components were miscible. In triplicate for each fixative/aroma chemical mixture, a porous non-woven cotton pad, 0.04 g/cm², available from Buckeye Technologies, Inc., (Buckeye paper) to approximately 2 inches×2 inches was placed in a shallow aluminum pan and weighed to four decimal places. 1.0 grams of each fixative/aroma chemical mixture was applied to a separate pad using a dropper pipet and weighed again. For controls without fixatives, each aroma chemical (0.10 g), in triplicate, was also separately applied to a preweighed pad and pan and weighed to four decimal places. All samples were left open to the air at room temperature and 68% Relative humidity.

The weight of each sample was recorded at selected time intervals after applying the compositions. For the fixative/aroma chemical mixtures, percent weight loss was calculated as:

% Weight loss=(Wso−Wsf)/(0.1×(Wso−Wt))×100

for the aroma chemicals by themselves (without fixative), percent weight loss was calculated as:

% Weight loss=(Wso−Wsf)/(Wso−Wt)×100

In both formulas:

Wso is the weight of the sample including pad and aluminum pan at time zero,

Wsf is the weight of the sample including pad and aluminum pan after the lapsed time,

Wt is the weight of the pad and aluminum pan (tare weight).

The average percent weight loss for triplicate samples are given in the Tables 1 through 4. The calculation of percent weight loss for the fixative/aroma chemical mixtures assumes that the fixative does not evaporate over the duration of the test.

TABLE 1
Effect of Fragrance Fixative on Evaporation Rate of Isoamyl Acetate
% weight loss

| | | | TMPD Di | | | TMPD |
|---|---|---|---|---|---|---|
| Time, | Without | | 2Ethyl | | TEXANOL | Di- |
| minutes | Fixative | TXIB | hexanoate | DEP | laurate | laurate |
| 0 | 0 | 0 | 0 | 0 | 0 | |
| 15 | 57.0001 | 18.5664 | 15.3172 | 10.6703 | 9.1116 | 8.1836 |
| 30 | 91.9451 | 29.7683 | 28.3977 | 24.0795 | 23.9169 | 25.0108 |
| 45 | 94.4484 | 41.3356 | 40.1964 | 36.5661 | 37.4316 | 38.8482 |
| 60 | 94.9323 | 51.5938 | 50.9459 | 48.1621 | 49.4562 | 50.7792 |
| 75 | 95.2313 | 59.2325 | 58.9665 | 56.3239 | 58.1023 | 59.2593 |
| 90 | 95.3847 | 65.445 | 65.6731 | 63.4943 | 65.4903 | 66.5883 |
| 105 | 95.5682 | 71.2327 | 71.3944 | 69.6084 | 71.4202 | 72.3405 |
| 120 | 95.8539 | 75.9753 | 75.9644 | 74.5978 | 76.2246 | 76.811 |

TABLE 2
Effect of Fragrance Fixative on Evaporation Rate of Benzyl Alcohol
% weight loss

| | | | TMPD Di | | | TMPD |
|---|---|---|---|---|---|---|
| Time, | Without | | 2Ethyl | | TEXANOL | Di- |
| hours | Fixative | TXIB | hexanoate | DEP | laurate | laurate |
| 0 | 0 | 0 | 0 | 0 | 0 | |
| 2 | 8.6376 | 6.1602 | 8.2416 | 1.678 | 8.1598 | 9.2648 |
| 4 | 17.8394 | 8.5449 | 11.883 | 2.4345 | 11.8085 | 14.7233 |
| 6 | 38.2846 | 25.1712 | 28.4654 | 18.362 | 28.3934 | 31.5308 |
| 7 | 45.3355 | 31.9938 | 35.0846 | 24.0548 | 34.8615 | 38.1147 |
| 24 | 91.4374 | 65.6431 | 68.4143 | 47.7117 | 68.2303 | 74.0452 |

TABLE 3
Effect of Fragrance Fixative on Evaporation Rate of Citronellal
% weight loss

| | | | TMPD Di | | | TMPD |
|---|---|---|---|---|---|---|
| Time, | Without | | 2Ethyl | | TEXANOL | Di- |
| hours | Fixative | TXIB | hexanoate | DEP | laurate | laurate |
| 0 | 0 | 0 | 0 | 0 | 0 | 0 |
| 3 | 34.6856 | 5.6716 | 6.8918 | 6.1143 | 8.8166 | 6.4056 |
| 4 | 47.6782 | 8.8873 | 10.502 | 9.4512 | 12.7093 | 9.8068 |
| 6 | 67.6466 | 13.7288 | 15.8518 | 14.7678 | 19.4299 | 15.5522 |
| 8 | 77.4317 | 18.0064 | 20.8069 | 19.6249 | 24.6866 | 19.8118 |
| 24 | 86.084 | 45.8937 | 47.3208 | 46.9007 | 53.3988 | 47.1524 |
| 32 | 86.7127 | 58.3945 | 56.6765 | 56.5397 | 62.3483 | 55.9025 |
| 48 | 86.7156 | 74.6403 | 66.2615 | 67.1336 | 70.3331 | 65.3129 |

TABLE 4
Effect of Fragrance Fixative on Evaporation Rate of Linalool
% weight loss

| | | | TMPD | | | TMPD |
|---|---|---|---|---|---|---|
| Time, | Without | | Di 2Ethyl | | TEXANOL | Di- |
| hours | Fixative | TXIB | hexanoate | DEP | laurate | laurate |
| 0 | 0 | 0 | 0 | 0 | 0 | 0 |
| 2 | 13.8251 | 0.7932 | 2.0472 | 1.9191 | 2.251 | NA |
| 4 | 34.1037 | 4.5633 | 6.3068 | 6.4841 | 6.5545 | NA |
| 6 | 53.4174 | 9.0275 | 11.1608 | 11.9423 | 11.123 | NA |
| 8 | 71.1828 | 13.2277 | 15.7504 | 16.8048 | 15.6582 | NA |
| 24 | 93.9987 | 41.5042 | 44.3455 | 47.5019 | 43.2999 | NA |
| 30 | 93.189 | 50.8302 | 52.0721 | 55.6721 | 50.7482 | NA |
| 48 | 84.7192 | 65.1172 | 60.063 | 65.0654 | 59.6532 | NA |
| 55 | 91.8785 | 80.1644 | 72.0492 | 78.363 | 72.034 | NA |

### EXAMPLE 2

50.0 grams of paraffin wax, 5.0 grams of stearic acid and 2.5 grams of TMPD di-2-ethylhexanoate (TMPD di-2-EH) and 1.5 g of a fragrance oil (“Finess Type, Bqt. 43135” available from Intarome Flavor and Fragrance Corp) were placed into a covered glass container. The container was heated in an oven until the oven temperature was 80° C. to melt all ingredients, and the contents were mixed until homogeneous. Melted contents were then poured into candle molds (containing wicks) and allowed to solidify. Solidified candles are refrigerated to shrink and allow separation from the mold.

Having described the invention in detail, those skilled in the art will appreciate that modifications may be made to the various aspects of the invention without departing from the scope and spirit of the invention disclosed and described herein. It is, therefore, not intended that the scope of the invention be limited to the specific embodiments illustrated and described but rather it is intended that the scope of the present invention be determined by the appended claims and their equivalents. Moreover, all patents, patent applications, publications, and literature references presented herein are incorporated by reference in their entirety for any disclosure pertinent to the practice of this invention, except that such incorporations by reference shall not change the meaning of any terms defined in this application.

What is claimed is:

1. A fragrance composition comprising at least one fragrance compound and at least one diester of TMPD.

2. The fragrance composition of claim 1 wherein at least one diester of TMPD is selected from TMPD di-2-ethyl hexanoate, 2,2,4-trimethyl-1,3-pentanediol di-isobutyrate, TEXANOL laurate, and TMPD di-laurate.

3. The fragrance composition of claim 1 wherein at least one diester of TMPD is TMPD di-2-ethyl hexanoate.

4. The fragrance composition of claim 2 wherein the amount of diester of TMPD is from about 0.5 to about 15 weight %, based on the total weight of the fragrance composition.

5. A scented composition comprising the fragrance composition of claim 2 and at least one additional component.

6. The scented composition of claim 2, wherein at least one additional component is a wax.

7. The scented composition of claim 2, wherein the wax is present in an amount of at least about 50% by weight based on the total weight of the scented composition.

8. The scented composition of claim 2, wherein the wax is present in an amount of at least about 80% by weight based on the total weight of the scented composition.

9. A scented composition comprising:
at least one fragrance compound;
at least one wax; and
at least one diester of TMPD.

10. The scented composition of claim 9 wherein at least one diester of TMPD is selected from TMPD di-2-ethyl hexanoate, 2,2,4-trimethyl-1,3-pentanediol di-isobutyrate, TEXANOL laurate, and TMPD di-laurate.

11. The scented composition of claim 9 wherein at least one diester of TMPD is TMPD di-2-hexanoate.

12. The scented composition of claim 9 wherein the amount of diester of TMPD is from about 0.5 to about 10 weight %, based on the total weight of the scented composition.

13. The scented composition of claim 9 wherein the amount of diester of TMPD is from about 2.0 to about 5.0 weight %, based on the total weight of the scented composition.

14. The scented composition of claim 9 wherein one or more waxes are present in an amount sufficient to render the scented composition a solid at room temperature.

15. The scented composition of claim 14, wherein the one or more waxes are present in an amount of at least about 50% by weight based on the total weight of the scented composition.

16. The scented composition of claim 14, wherein the one or more waxes are present in an amount of at least about 80% by weight based on the total weight of the scented composition.

17. A solid scented composition comprising:
between about 0.5% and about 10%, based on the total weight of the fragrance composition, of at least one fragrance compound;
one or more waxes present in an amount sufficient to render the solid fragrance composition a solid at room temperature and standard pressure; and
between about 0.5% and about 15%, based on the total weight of the fragrance composition, of at least one diester of TMPD.

18. An article comprising the solid scented composition of claim 17.

19. The article of claim 18, wherein the article is a candle or a solid air freshener.

20. An article comprising the fragrance composition of claim 1.

21. An article comprising the scented composition of claim 9.

22. A method of imparting an aroma to a location, comprising delivering the fragrance composition of claim 1 to the location.

23. A method of imparting an aroma to a location, comprising delivering the scented composition of claim 9 to the location.

24. A method of imparting an aroma to a location, comprising delivering the solid scented composition of claim 17 to the location.

25. A method for formulating a fragrance composition, comprising combining at least one fragrance compound with at least one diester of TMPD.

26. A method for formulating a scented composition, comprising combining at least one fragrance compound, at least one wax, and at least one diester of TMPD.
